(19) United States
(12) Patent Application Publication
(10) Pub. No.: US 2026/0122140 A1
(43) Pub. Date: Apr. 30, 2026

(54) CONNECTION OF A MEDICAL DEVICE TO A LOCAL SERVER

(71) Applicant: Maquet Critical Care AB, Solna (SE)

(72) Inventor: Jonas ANDERSSON, Tyresö (SE)

(21) Appl. No.: 19/431,364

(22) Filed: Dec. 23, 2025

Related U.S. Application Data
(63) Continuation of application No. PCT/EP2024/067916, filed on Jun. 26, 2024.

(30) Foreign Application Priority Data
Jun. 29, 2023 (SE) 2350810-4

(51) Int. Cl. H04L 67/141; A61B 5/00; H04L 67/12

(52) U.S. Cl. CPC H04L 67/141; A61B 5/002; H04L 67/12

(57) ABSTRACT

The present disclosure relates to a method for connecting a first medical device to a local server. The method comprises transmitting, to an external server, a request for information, comprising a customer ID associated with the local server; receiving, from the external server, a plurality of device IDs associated with the customer ID, wherein each device ID is associated with a respective one of a plurality of medical devices; receiving a connection request from the first medical device, comprising a first device ID associated with the first medical device; comparing the first device ID with the plurality of device IDs received from the external server; and upon the first device ID corresponding to a device ID of the plurality of device IDs, allowing the connection request to the local server.

### CROSS-REFERENCE TO RELATED APPLICATIONS

This application is a continuation under 35 U.S.C. § 120 of International Application No. PCT/EP2024/067916, filed Jun. 26, 2024, which claims priority to Swedish Application No. 2350810-4, filed Jun. 29, 2023, under 35 U.S.C. § 119 (a). Each of the above-referenced patent applications is incorporated by reference in its entirety.

### TECHNICAL FIELD

The present invention relates to medical device connectivity, and more specifically to technologies for connecting a medical device to a local server comprising an environment for running an application using data generated by the medical device.

### BACKGROUND

Medical device connectivity has become an increasingly important aspect of healthcare technology, primarily due to the evolving need for real-time transfer and monitoring of patient data as well as equipment data. Medical devices, such as monitors, ventilators, extracorporeal membrane oxygenation devices, infusion pumps, and similar devices, may establish and maintain a connection through which data are transferred to a local server in a healthcare setting. The data transfer can be employed for various applications, including applications for patient monitoring, diagnostics, device status monitoring, and device control.

For data privacy and security purposes, it is of importance that the medical device is authenticated and allowed to connect to the local server. This ensures that the transferred data are secured, thereby reducing the risk for unauthorised access and potential data breaches.

However, the existing methods of connecting the medical devices to the local server typically involve manual configuration processes, which can be time-consuming with a potential for errors, especially when a large number of devices are involved. Therefore, there exists a need for an improved technology that can simplify the connection process and enhance the overall security of medical device data transfer.

### SUMMARY

In view of the above, it is thus an object of the present invention to overcome or at least mitigate some of the problems discussed above. In particular, it is an object to provide an improved technology for connecting a medical device to a local server.

Hence, according to a first aspect, there is provided a method for connecting a first medical device to a local server, wherein the local server comprises an environment for running an application using data generated by the first medical device. The method comprises transmitting a request for information to an external server, wherein the request comprises a customer ID associated with the local server, and receiving, from the external server, a plurality of device IDs associated with the customer ID. Each device ID is associated with a respective one of a plurality of medical devices. The method further comprises receiving a connection request from the first medical device, wherein the connection request comprises a first device ID associated with the first medical device, comparing the first device ID with the plurality of device IDs received from the external server, and, upon the first device ID corresponding to a device ID of the plurality of device IDs, allowing the connection request to the local server.

According to a second aspect, a system is provided, comprising an external database configured to hold and update information pertaining to a plurality of medical devices, wherein the information comprises a plurality of device IDs, each of which being associated with a respective one of the plurality of medical devices, and a customer ID associated with the plurality of device IDs. The system further comprises an external server that is connected to the external database and configured to receive the customer ID and the plurality of device IDs from the external database, as well as a local server that is connected to the external server, wherein the local server is associated with the customer ID and configured to transmit, to the external server, a request for information. The request for information comprises the customer ID. The external server is configured to transmit, to the local server, the plurality of device IDs associated with the customer ID. The local server comprises an environment for running an application using data from a first medical device and is configured to receive a connection request from the first medical device, wherein the connection request comprises a first device ID associated with the first medical device. The local server is further configured to compare the first device ID with the plurality of device IDs received from the external server and, upon the first device ID corresponding to a device ID of the plurality of device IDs, allow the connection request.

Medical devices that communicate with a local server are typically found in healthcare settings. For data privacy and security purposes, it is of interest to authenticate the medical devices to reduce the risk of unauthorised access and potential data breaches. The present invention beneficially provides a technology for handling a request from a first medical device to establish a communicative connection to the local server. This request may, for example, form part of a configuration process or an onboarding process in which the first medical device is connected to a local network within the healthcare setting. The connection request comprises an identifier (i.e., device ID) associated with the medical device that requests to connect to the local server. The device ID may be compared with a predetermined list of authorised device IDs received from an external server. The first medical device may then be considered authorised if its associated device ID is found on the list received from the external server. In this way, illegitimate and possibly malicious attempts to gain unauthorised access to the local server may be detected. Further, the configuration or onboarding of new equipment may be simplified, as the local server is configured to automatically authenticate the medical device sending the request.

In this context, it may be useful to refer to the entity responsible for the healthcare setting, in which the medical devices may be installed, as the ‘customer’, and the entity providing the medical devices as the ‘provider’. In some examples, the provider may hold and update the external database and the external server, whereas the local server and the first medical device may be located within the healthcare setting, typically on a local network protected by a firewall. The present invention allows for information that is kept and updated by the provider to be employed by the customer to authenticate a device requesting to connect to the customer's local server. In this way, the onboarding process, in which a medical device is connected to the local server, may be simplified, and require less manual configuring of the system.

The external database may comprise information about a plurality of customers, and list all medical devices purchased by, or delivered to, the respective customer. The external server may be configured to receive a subset of the information stored in the external database, i.e., a list of device IDs associated with a specific customer ID. The external server may hence be up to date with which medical devices (device IDs) the provider believes are associated with a customer (customer ID) to which the local server is associated. These device IDs may be considered authenticated and allowed to connect to the local server.

In this way, the acts involved in connecting the first medical device to the local server may be distributed across different nodes of the system, wherein the list of authorised device IDs may be maintained by an external entity outside the healthcare setting, and wherein the evaluation of the connection request, in which the device ID of the first medical device requesting to connect, may be performed within the healthcare setting.

In some embodiments, the local server and the first medical device may be arranged in a local network, such as a local network of a healthcare setting. Further, the external server may be arranged outside the local network, such as on a cloud-based platform or being provided as a bare metal server on an external network. The local network may typically be protected by a firewall to increase data privacy and security.

In some embodiments, the request for information may be transmitted periodically to the external server. This allows for the local server to be regularly provided with up-to-date information and maintain an accurate list of authorised device IDs. The periodical updating beneficially allows for the local server to maintain fresh data without necessarily requiring complex event-handling code. It will however be appreciated that in alternative, or additional examples, the request for information may be triggered by a connection request received from a medical device.

The connection request may be received upon installation, or onboarding, of the medical device. The connection request may, for example, be sent in response to the medical device being turned on and connected to the local network for the first time. The connection request may be initiated by an operator inputting a command for sending the connection request, or automatically by the medical device upon connection to the local network. It will however be appreciated that the connection request may be sent in response to other events than installation. The connection request may, for example, be sent in response to the medical device being introduced into a medical treatment area, or in response to the medical device being powered into an active power state.

In some embodiments, the local server may allow the connection request by sharing a communication certificate with the medical device. The communication certificate may be encrypted, and the sharing of the certificate referred to as a handshake. A handshake procedure may thus be a process by which two devices (i.e., the medical device and the local server) agree upon the parameters of their interaction before the medical device sends data to the local server based on the agreed-upon parameters of their interaction.

In some embodiments, the local server may be assigned the customer ID upon installation of the local server. The customer ID may be provided from the external server, for example as metadata added to a communication certificate. The assigned customer ID may then be used by the local server when requesting information from the external server, including an updated list of authorised device IDs, as previously discussed. A local server may be configured with one or more customer IDs.

The medical device may be configured to transmit information to the local server. The information may comprise personal identifiable information, PII, and non-PII. PII may be understood as data that can be used on its own or with other information to identify a patient and typically include patient identification information such as name, ID number and the like. The PII may further relate to treatment information, such as oxygen concentration, tidal volume, blood flow rate, physiological responses, administered medications, and duration of the treatment. Non-PII information generally refers to information that cannot be used to identify, directly or indirectly, an individual, such as aggregate statistics, device information relating to make, model, software version, maintenance logs, and general usage information such as number of times a particular device has been used over a period of time. The non-PII information may also include de-identified data from which all identifying information has been removed, such as oxygen levels, heart rates, or blood pressure readings that are not tied to a specific individual.

In some embodiments, the medical device may be configured to transmit information comprising both PII and non-PII to the local server. The PII and the non-PII may be used by one or more applications run in the application environment on the local server. The applications may, for example, be clinical applications, applications for decision support, or applications assisting in optimising the operation of the medical devices. Preferably, the local server is arranged on a local network within the healthcare setting, which is protected by a firewall to ensure data privacy and security.

In some embodiments, the local server may be configured to filter the information from the medical device to remove the PII and transmit the filtered information, comprising the non-PII, to the external server. The PII may, for example, be used by technical staff for maintenance and service of the medical devices installed at the healthcare setting. By filtering out the PII information before sending the information to the external server, the external server may be arranged outside the local network (i.e., outside the firewall), such as in a cloud-based environment. This allows for a cloud-based design or a Client-Server Architecture to be used for managing the medical devices, while ensuring that PII is kept within the boundaries of the local network of the healthcare setting to comply with data protection regulations.

In some embodiments, the external server may be configured to transmit the filtered information, comprising the non-PII, to the external database. The transmitted information may, for example, comprise information pertaining to software versions and maintenance logs, which may be transmitted by the external server to update the device information in the external database. In this way, the provider may have access to updated information about the medical devices installed at the customer's facilities.

In some embodiments, the external server may be configured to periodically receive the customer ID and the plurality of device IDs from the external database. The external database may be configured to push the customer ID and the plurality of device IDs to the external database on a regular basis, such as once a day, or once a week, or upon changes in the information pertaining to the information in the external database, to ensure that the external server is up to date. Any changes in the device list, for example corresponding to the customer acquiring a new device from the provider, may be reflected in an update of the list of device IDs provided to the external server.

The external database may be a database held and maintained by the provider of the medical devices, such as a database used by an enterprise to manage business operations and customer relations. The external database may comprise information about a plurality of customers, and list all medical devices purchased by, or delivered to, each of the customers.

The external server may be controlled by the provider and may be set up to communicate with one or more local servers. Hence, the external server may receive information from the external database relating to one or more local servers (customer IDs), depending on which local server(s) the external server is configured to communicate with. The external server may be arranged outside the healthcare setting, i.e., outside any firewall protecting the customer's local network. In some examples, the external server is provided on a cloud computing platform. The local server may comprise an environment for running applications assisting technical staff performing service and maintenance of the medical devices installed in the customer's facility, such as a healthcare setting. This is also referred to as ‘fleet management’ and may utilise equipment data received from the local server.

The local server may be arranged in a local network within the healthcare facility and form a hub, to which the connected medical devices can send information. A local server in the local network may hence be entrusted with sensitive information, such as the above-mentioned PII. The local server may be an application server, comprising an application runtime environment for applications using information from the medical devices. It may be beneficial to run the applications on the local server instead of on the medical devices, since the local server may have a higher computing capacity compared to the medical device.

The medical device may refer to a product or piece of equipment used in healthcare for the diagnosis, monitoring, treatment, or alleviation of disease or injury. Typically, the medical device is able to connect to the local server over a network for the purpose of sharing data and controlling the operation of the device. Examples of medical devices that have connectivity capabilities include patient monitors, infusion pumps, ventilators, and extracorporeal oxygenation machines.

According to a third aspect, a local server comprising an environment for running an application using data from a first medical device, is provided. The local server is configured to transmit, to an external server, a request for information, wherein the request for information comprises a customer ID associated with the local server. Further, the local server is configured to receive, from the external server and in response to the request for information, a plurality of device IDs associated with the customer ID, wherein each device ID is associated with a respective one of a plurality of medical devices, and to receive a connection request from the first medical device, wherein the connection request comprises a first device ID associated with the first medical device. The local server may then compare the first device ID with the plurality of device IDs received from the external server, and, upon the first device ID corresponding to a device ID of the plurality of device IDs, allow the connection request.

According to a fourth aspect, one or more non-transitory computer-readable media are provided, storing instructions executable by one or more processors. The instructions, when executed, cause the one or more processors to perform operations comprising:
transmitting, to an external server, a request for information, wherein the request for information comprises a customer ID associated with a local server comprising an environment for running an application using data from a first medical device connected to the local server;
in response to the request for information, receiving, from the external server, a plurality of device IDs associated with the customer ID, wherein each device ID is associated with a respective one of a plurality of medical devices;
receiving a connection request from the first medical device, the connection request comprising a first device ID associated with the first medical device;
comparing the first device ID with the plurality of device IDs received from the external device; and
upon the first device ID corresponding to a device ID of the plurality of device IDs, allowing the connection request to the local server.

In different words, the above object is achieved by a computer program product comprising a computer-readable medium with computer code instructions adapted to carry out the method of the first aspect when executed by a device having processing capability.

The third and fourth aspects may generally have the same features and advantages as the first and second aspects. It is further noted that the invention relates to all possible combinations of features unless explicitly stated otherwise.

### BRIEF DESCRIPTION OF THE DRAWINGS

The above, as well as additional objects, features and advantages of the present invention, will be better understood through the following illustrative and non-limiting detailed description of preferred embodiments of the present invention, with reference to the appended drawings, where the same reference numerals will be used for similar elements, wherein:

FIG. 1 shows a local server and a system according to some embodiments;

FIG. 2 shows a method for connecting a medical device to a local server according to some embodiments; and

FIG. 3 shows the transmission of information generated by a medical device to a local server according to an embodiment.

### DETAILED DESCRIPTION

The present invention will now be described more fully hereinafter with reference to the accompanying drawings, in which embodiments of the invention are shown.

For data privacy and security reasons, a medical device that communicates with a local server in, for example, a healthcare setting, needs to be authenticated. According to the present invention, such an authentication process typically requires that the medical device sends a connection request to the local server, wherein the request for information comprises an identifier, also referred to as a device ID, of the medical device. The device ID may for example be a serial number of the medical device, or any other identifier that may be unique to the device. The local server may validate the request by checking if the device ID corresponds to a device ID received from an external server and send a response indicating whether the authentication was successful.

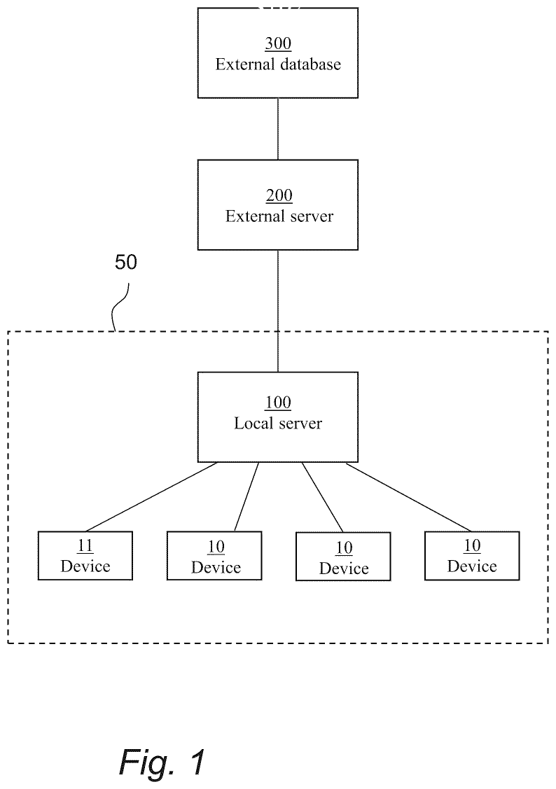

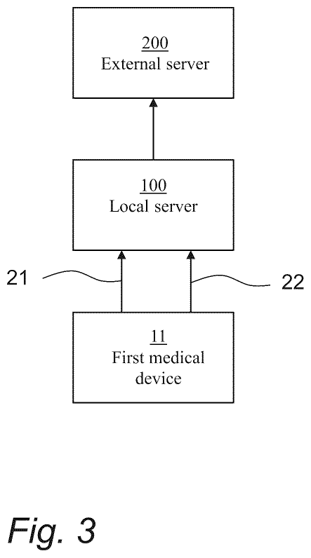

FIG. 1 shows a local server 100 according to an embodiment, comprising an environment for running an application that uses data from one or several medical devices 10 connected to the local server 100. The local server 100 is arranged on a local network (local area network) 50 of a healthcare setting, such as a hospital, and is associated with an identifier, or customer ID, by which the local server 100 may be identified. The communication within the local network may, for example, be wireless. The local server 100 and the plurality of medical devices 10 may be configured to share information within the local network, including clinical data relating to patient and treatment information, and equipment data relating to, model, software version, maintenance logs, and the like. The communication of data between the medical devices 10 and the local server 100 will be discussed in greater detail in connection with FIG. 3.

The local server 100 may comprise an application runtime environment, comprising a container management platform such as a local Kubernetes platform. The environment may also be referred to as a Control Centre and may form a platform for various applications providing real-time information to a user and assisting in analytics, reporting and maintenance of the connected devices.

The local server 100 is further connected to an external server 200, which may be a cloud-based server or bare metal server arranged outside the local network 50. The external server 200 may comprise a list of device IDs associated with the customer ID of the local server 100 and is configured to transmit the list of device IDs to the local server 100 periodically or upon request by the local server 100. The external server 200 may be arranged to communicate with a specific local server 100 only, or a plurality of local servers. In the latter case, the external server 200 may comprise information pertaining to a plurality of customer IDs and their associated device IDs. When receiving a request for information from one of the associated local servers, the external server may request the local server's customer ID and respond with sending the device IDs associated with the customer ID to the local server.

The external server 200 may be controlled by an entity different from the one controlling the medical devices 10 and the local server 100. The entity controlling the external server 200 may, for instance, be a provider of the medical devices 10 (and, in some examples, the local server 100), whereas the entity controlling the healthcare setting, in which the medical devices 10 and the local server 100 are installed, may be referred to as a customer.

The external server 200 may further be communicatively connected to an external database 300. The external database 300 may be held and maintained by the provider and may for instance be a database in an enterprise software system for managing business operations and customer relations. The provider may use the external database 300 to record information about the customer and the medical devices 10 delivered to the customer. The external database may for example be a relational database with a first table for customer data, such as customer IDs, and another table for order data, such as device IDs. These two tables may be related via the customer ID, associating each customer with a list of devices purchased by the customer.

In the present example, the external database 300 is configured to transmit a plurality of device IDs associated the customer ID to the external server 200 on a regular basis, such as daily or weekly. In further examples, the plurality of device IDs may be pushed to the external server 200 in response to the information being updated in the external database 300.

It will be appreciated that the external database 300 may be communicatively connected to a plurality of external servers 200, and that each of the plurality of external servers 200 may be configured to communicate with one or more local servers 100 as described above.

Hence, according to the present examples, the external database 300 may be configured to push information about approved medical devices to the external server 200, which may be queried for updated information by the local server 100. The updated information may then be used by the local server 100 to authenticate medical devices 10 that requests to connect to the server.

In the present example, example of medical devices may include be ventilators or extracorporeal oxygenators, monitors, sensors, and other types of healthcare equipment.

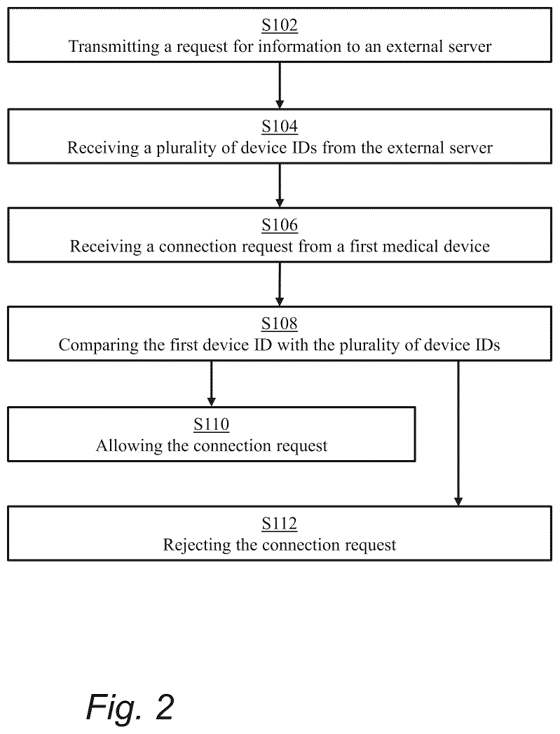

FIG. 2 shows by way of example a method for connecting a first medical device to a local server 100 as described above. According to the present example, a request for information is transmitted S102 to the external server. The request may in some examples be sent periodically, such as daily or weekly. In other examples, the request may be initiated by an operator. In further examples, the request may be sent upon installation of the local server. The request for information comprises a customer ID associated with the local server and, hence, the entity or customer controlling the healthcare setting.

A plurality of device IDs associated with the customer ID may be received S104 from the external server in response to the request for information. The plurality of device IDs may also be referred to as a list of authorised device IDs, i.e., a list of device IDs which are considered authentic and allowed to connect to the local server. As mentioned above, this list may be used to determine is a connection request should be allowed or not.

The method further comprises receiving S106 a connection request from the first medical device. The connection request may be sent upon the first medical device being connected to the local network on which the local server is arranged. The connection request comprises a first device ID associated with the first medical device.

This first device ID may then be compared S108 with the plurality of device IDs on the list received from the external server to validate the connection request. If the first device ID matches any of the device IDs from the external server, the local server may consider the connection request to be valid. As a result, the local server may allow S110 the connection request. If the first device ID does not match any of the listed device IDs received from the external server, the local server may deny S112 the connection request.

The server may allow S110 the connection request by sharing a communication certificate with the first medical device. The communication certificate may be used for securing future communication between the first medical device and the local server and may utilise a public-private key pair. The process in which the local server allows the connection request may also be referred to as a handshake, in which the local server presents the communication certificate to the medical device. The first medical device may then use the public key to encrypt a session key, which is sent to the local server. The local server may then use its private key to decrypt the session key. After this, both the first medical device and the local server have the same session key, which they can use to encrypt and decrypt communication between them.

A similar procedure may be performed upon installation of the local server to ensure encrypted communication with the external server. Similarly, encrypted communication may also take place between the external database and the external server.

It should be noted that although FIG. 2 shows the steps of the method as a sequence of successive steps, the steps need not be performed strictly in the shown order. Further, two or more steps may be performed simultaneously. For instance, the transmission of the request for information to the external server may be performed after receiving the connection request from the first medical device.

FIG. 3 illustrates the communication of information generated by the first medical device 11 after the connection request has been allowed, as discussed above. The information may hence be transmitted over a local network, to which the first medical device 11 and the local server 100 may be connected. The local network may be arranged within a healthcare setting and protected to outside networks by a firewall. As previously mentioned, the information may be used by various applications run on an application environment provided by the local server 100.

The information may comprise two main types of data: personal identifiable information (PII) 21, and non-PII 22. PII 21 may be understood as information that typically includes patient identification information, such as name, ID number and the lie, as well as treatment information, such as oxygen concentration, tidal volume, blood flow rate, physiological responses, and other type of data that on its own, or with other information, can be used to identify an individual. Naturally, this type of data is considered sensitive and need to be handled with care. Preferably, the PII 21 should stay within the healthcare setting and not be transmitted to, or accessible from, outside the local network of the healthcare setting. The PII 21 may typically be used by a healthcare provider to monitor, control, and optimise the healthcare delivered to the patient.

Non-PII 22, on the other hand, typically relates to information that cannot be used to identify an individual. Examples of such information include equipment data like make and model, software versions, maintenance information, and aggregate statistics on device usage and the like. This type of information is generally considered less sensitive from a patient integrity point of view and may be allowed to transmit and handle outside the information infrastructure system of the healthcare setting.

As illustrated in FIG. 3, the PII 21 and the non-PII 22 may be transmitted to the local server 100, where it may be used in various applications for assisting and optimising the healthcare delivered to the patient. The local server 100 may further be configured to filter the information to remove the PII 21 and transmit the filter information, comprising the non-PII 22 but no the PII 21, to the external server 200. In different words, the equipment data may be sent outside the healthcare setting, such as to a cloud-based server. This allows for technical staff to access and monitor data concerning the performance and status of the equipment. The equipment data may, for example, be used on a global level by the provider to analyse and optimise various medical devices installed at various customers, without risking accessing the more sensitive PII 21. Further, the present example allows the external server to access the non-PII without having direct access to the customer's local network.

Generally, the medical devices 10, 11, and/or the local server 100 may comprise circuitry which is configured to implement (using one or more non-transitory computer-readable media) the functionality described herein. Suitable processors for the execution of a program of instructions include, by way of example, both general and special purpose microprocessors, and the sole processor or one of multiple processors or cores, of any kind of computer. The processors can be supplemented by, or incorporated in, ASICs (application-specific integrated circuits). Those skilled in the art will understand that the above-described exemplary embodiments may be implemented in any suitable software, hardware, or firmware configuration or combination thereof. An exemplary hardware platform for implementing the exemplary embodiments may include, for example, an Intel x86 based platform with compatible operating system, a Windows OS, a Mac platform and MAC OS, a mobile device having an operating system such as iOS, Android, etc. In a further example, the exemplary embodiments of the above-described method may be embodied as a program containing lines of code stored on a non-transitory computer readable storage medium that, when compiled, may be executed on a processor or microprocessor.

Additionally, variations to the disclosed embodiments can be understood and effected by the skilled person in practising the claimed invention, from a study of the drawing, the disclosure, and the appended claims. Moreover, in the drawings and specification, there have been disclosed preferred embodiments and examples of the invention and, although specific terms are employed, they are used in a generic and descriptive sense only and not for the purpose of limitation. The scope of the invention is set forth in the following claims, in which the word ‘comprising’ does not exclude other elements or steps, and the indefinite article ‘a’ or ‘an’ does not exclude a plurality.

### CLAUSES

Clause 1. A method for connecting a first medical device (10) to a local server (100), wherein the local server comprises an environment for running an application using data generated by the first medical device, the method comprising:
   transmitting (S102), to an external server (200), a request for information, the request for information comprising a customer ID associated with the local server;
   receiving (S104), from the external server, a plurality of device IDs associated with the customer ID, wherein each device ID is associated with a respective one of a plurality of medical devices;
   receiving (S106) a connection request from the first medical device, the connection request comprising a first device ID associated with the first medical device;
   comparing (S108) the first device ID with the plurality of device IDs received from the external server; and
   upon the first device ID corresponding to a device ID of the plurality of device IDs, allowing (S110) the connection request to the local server.
Clause 2. The method according to clause 1, wherein the local server and the first medical device are arranged in a local network of a healthcare setting, and wherein the external server is arranged outside the local network.
Clause 3. The method according to clause 1 or 2, wherein the request for information is transmitted periodically to the external server.
Clause 4. The method according to any of the preceding clauses, wherein the connection request is received upon installation of the first medical device.
Clause 5. The method according to any of the preceding clauses, wherein allowing the connection request comprises sharing a communication certificate with the first medical device.
Clause 6. The method according to any of the preceding clauses, wherein the first medical device is a cardiac and/or respiratory support device.
Clause 7. A local server (100) comprising an environment for running an application using data from a first medical device (11), the local server being configured to:
   transmit, to an external server (200), a request for information, the request for information comprising a customer ID associated with the local server;
   receive, from the external server, a plurality of device IDs associated with the customer ID, wherein each device ID is associated with a respective one of a plurality of medical devices;
   receive a connection request from the first medical device, the connection request comprising a first device ID associated with the first medical device;
   compare the first device ID with the plurality of device IDs received from the external server; and
   upon the first device ID corresponding to a device ID of the plurality of device IDs, allow the connection request.
Clause 8. A system comprising:
   an external database (300) configured to hold and update information pertaining to a plurality of medical devices (10), the information comprising:
      a plurality of device IDs, wherein each device ID is associated with a respective one of the plurality of medical devices, and
      a customer ID associated with the plurality of device IDs;
   the system further comprising:
   an external server (200) connected to the external database and configured to receive the customer ID and the plurality of device IDs from the external database; and
   a local server (100) connected to the external server, wherein the local server is associated with the customer ID and configured to transmit, to the external server, a request for information, the request for information comprising the customer ID;
   wherein the external server is configured to, upon receiving the request for information from the local server, transmit, to the local server, the plurality of device IDs associated with the customer ID;
   wherein the local server comprises an environment for running an application using data from a first medical device (11);
   wherein the local server is further configured to receive a connection request from the first medical device, the connection request comprising a first device ID associated with the first medical device; and
   wherein the local server is further configured to compare the first device ID with the plurality of device IDs received from the external server and, upon the first device ID corresponding to a device ID of the plurality of device IDs, allow the connection request.
Clause 9. The system according to clause 8, wherein the local server is configured to receive information from the medical device, the information comprising personal identifiable information, PII, and non-PII, and to filter the information to remove the PII and transmit the filtered information to the external server.
Clause 10. The system according to clause 9, wherein the external server is configured to transmit the filtered information comprising the non-PII to the external database.
Clause 11. The system according to clause 9 or 10, wherein the non-PII comprises equipment data pertaining to the medical device.
Clause 12. The system according to any of clauses 8-11, wherein the external server is configured to periodically receive the customer ID and the plurality of device IDs from the external database.
Clause 13. The system according to any of clauses 8-12, wherein the local server and the first medical device are arranged in a local network, and wherein the external database and the external server are arranged externally to the local network.
Clause 14. One or more non-transitory computer-readable media storing instructions executable by one or more processors, wherein the instructions, when executed, cause the one or more processors to perform operations comprising:
   transmitting (S102), to an external server, a request for information, the request for information comprising a customer ID associated with a local server comprising an environment for running an application using data from a first medical device connected to the local server;
   receiving (S104), from the external server, a plurality of device IDs associated with the customer ID, wherein each device ID is associated with a respective one of a plurality of medical devices;
   receiving (S106) a connection request from the first medical device, the connection request comprising a first device ID associated with the first medical device;
   comparing (S108) the first device ID with the plurality of device IDs received from the external device; and
   upon the first device ID corresponding to a device ID of the plurality of device IDs, allowing (S110) the connection request to the local server.

1. A method for connecting a first medical device to a local server, wherein the local server comprises an environment for running an application using data generated by the first medical device, the method comprising:
transmitting, to an external server, a request for information, the request for information comprising a customer ID associated with the local server;
receiving, from the external server, a plurality of device IDs associated with the customer ID, wherein each device ID is associated with a respective one of a plurality of medical devices;
receiving a connection request from the first medical device, the connection request comprising a first device ID associated with the first medical device;
comparing the first device ID with the plurality of device IDs received from the external server; and
upon the first device ID corresponding to a device ID of the plurality of device IDs, allowing the connection request to the local server.

2. The method according to claim 1, wherein the local server and the first medical device are arranged in a local network of a healthcare setting, and wherein the external server is arranged outside the local network.

3. The method according to claim 1, wherein the request for information is transmitted periodically to the external server.

4. The method according to claim 1, wherein the connection request is received upon installation of the first medical device.

5. The method according to claim 1, wherein allowing the connection request comprises sharing a communication certificate with the first medical device.

6. The method according to claim 1, wherein the first medical device is a cardiac and/or respiratory support device.

7. A local server comprising an environment for running an application using data from a first medical device, the local server being configured to:
transmit, to an external server, a request for information, the request for information comprising a customer ID associated with the local server;
receive, from the external server, a plurality of device IDs associated with the customer ID, wherein each device ID is associated with a respective one of a plurality of medical devices;
receive a connection request from the first medical device, the connection request comprising a first device ID associated with the first medical device;
compare the first device ID with the plurality of device IDs received from the external server; and
upon the first device ID corresponding to a device ID of the plurality of device IDs, allow the connection request.

8. A system comprising:
an external database configured to hold and update information pertaining to a plurality of medical devices, the information comprising:
a plurality of device IDs, wherein each device ID is associated with a respective one of the plurality of medical devices, and
a customer ID associated with the plurality of device IDs;
the system further comprising:
an external server connected to the external database and configured to receive the customer ID and the plurality of device IDs from the external database; and
a local server connected to the external server, wherein the local server is associated with the customer ID and configured to transmit, to the external server, a request for information, the request for information comprising the customer ID;
wherein the external server is configured to, upon receiving the request for information from the local server, transmit, to the local server, the plurality of device IDs associated with the customer ID;
wherein the local server comprises an environment for running an application using data from a first medical device;
wherein the local server is further configured to receive a connection request from the first medical device, the connection request comprising a first device ID associated with the first medical device; and
wherein the local server is further configured to compare the first device ID with the plurality of device IDs received from the external server and, upon the first device ID corresponding to a device ID of the plurality of device IDs, allow the connection request.

9. The system according to claim 8, wherein the local server is configured to receive information from the medical device, the information comprising personal identifiable information, PII, and non-PII, and to filter the information to remove the PII and transmit the filtered information to the external server.

10. The system according to claim 9, wherein the external server is configured to transmit the filtered information comprising the non-PII to the external database.

11. The system according to claim 9, wherein the non-PII comprises equipment data pertaining to the medical device.

12. The system according to claim 8, wherein the external server is configured to periodically receive the customer ID and the plurality of device IDs from the external database.

13. The system according to claim 8, wherein the local server and the first medical device are arranged in a local network, and wherein the external database and the external server are arranged externally to the local network.

14. One or more non-transitory computer-readable media storing instructions executable by one or more processors, wherein the instructions, when executed, cause the one or more processors to perform operations comprising:
transmitting, to an external server, a request for information, the request for information comprising a customer ID associated with a local server comprising an environment for running an application using data from a first medical device connected to the local server;
receiving, from the external server, a plurality of device IDs associated with the customer ID, wherein each device ID is associated with a respective one of a plurality of medical devices;
receiving a connection request from the first medical device, the connection request comprising a first device ID associated with the first medical device;
comparing the first device ID with the plurality of device IDs received from the external device; and
upon the first device ID corresponding to a device ID of the plurality of device IDs, allowing the connection request to the local server.
